SEQUENCE LISTING (30 sequences)

SEQ ID NO: 1 - DNA, 36 bp, Artificial Sequence
  PCR primer
aaaaggatcctagtaaaagggagtgtacgaccagtg

SEQ ID NO: 2 - DNA, 57 bp, Artificial Sequence
  PCR primer
ggggtcgcgaaaaaaggggggattattagtaatctatacatgttaacattcctccac

SEQ ID NO: 3 - DNA, 46 bp, Artificial Sequence
  PCR primer
cccccttttttcgcgaccccacttcttgcacttgcagaaggtggac

SEQ ID NO: 4 - DNA, 37 bp, Artificial Sequence
  PCR primer
aaaaggatcctctaaattctgcaatatatgccccccc

SEQ ID NO: 5 - DNA, 28 bp, Artificial Sequence
  PCR primer
ataacaggatatatgctctctgacgcgg

SEQ ID NO: 6 - DNA, 28 bp, Artificial Sequence
  PCR primer
gatcatcactcattttaaacatggggcc

SEQ ID NO: 7 - DNA, 41 bp, Artificial Sequence
  PCR primer
aaaaggatcccagacactataatagctttaggtggtacccc

SEQ ID NO: 8 - DNA, 58 bp, Artificial Sequence
  PCR primer
ggggaggcctaaaaagggggattataaaaagtagttgaaatatgaaggtttaaggttg

SEQ ID NO: 9 - DNA, 48 bp, Artificial Sequence
  PCR primer
ccccctttttaggcctccccatatccaatgaacttagacccatggctg

SEQ ID NO: 10 - DNA, 49 bp, Artificial Sequence
  PCR primer
aaaaggatccgtgttataatgtaaatataaataaataggactagaggcg

SEQ ID NO: 11 - DNA, 28 bp, Artificial Sequence
  PCR primer
taccaccttctttcacgcttggctgcgg

SEQ ID NO: 12 - DNA, 28 bp, Artificial Sequence
  PCR primer
tatttaaagaggcattatcaccagagcg

SEQ ID NO: 13 - DNA, 39 bp, Artificial Sequence
  PCR primer
aaaaggatccgctttaaaatttggaaagaggaagttgtg

SEQ ID NO: 14 - DNA, 56 bp, Artificial Sequence
  PCR primer
ggggaggcctaaaaagggggttagaaatctttaaaaatttctctatagagcccatc

SEQ ID NO: 15 - DNA, 57 bp, Artificial Sequence
  PCR primer
ccccctttttaggcctccccggtaaaagacctaaactccaagggtggaggctaggtc

SEQ ID NO: 16 - DNA, 48 bp, Artificial Sequence
  PCR primer
aaaaggatcccccattgtggagaatattccaaagaagaaaataattgc

SEQ ID NO: 17 - DNA, 31 bp, Artificial Sequence
  PCR primer
cagaaggcaagaatgtattaagcggaaatgc

SEQ ID NO: 18 - DNA, 31 bp, Artificial Sequence
  PCR primer
cttcccattatagctcttattcacattaagc

SEQ ID NO: 19 - DNA, 43 bp, Artificial Sequence
  PCR primer
aaaaggatcctattataacagtcaaacccaataaaatactggg

SEQ ID NO: 20 - DNA, 54 bp, Artificial Sequence
  PCR primer
ggggaggcctaaaaagggggttaatccatttgtattttctcccttcataatgcc

SEQ ID NO: 21 - DNA, 57 bp, Artificial Sequence
  PCR primer
ccccctttttaggcctcccctttattttgcatgcttatataataaattatggctgcg

SEQ ID NO: 22 - DNA, 40 bp, Artificial Sequence
  PCR primer
aaaaggatccgcttttccttcttttacaagatttaaagcc

SEQ ID NO: 23 - DNA, 26 bp, Artificial Sequence
  PCR primer
cacttttatttatcaagctgtaggcc

SEQ ID NO: 24 - DNA, 25 bp, Artificial Sequence
  PCR primer
tataccttttgaacctaggaaaggc

SEQ ID NO: 25 - DNA, 43 bp, Artificial Sequence
  PCR primer
aaaaggatccgcctcttctgtattatgcaaggaaagcagctgc

SEQ ID NO: 26 - DNA, 59 bp, Artificial Sequence
  PCR primer
ggggaggcctaaaaagggggtatataaaataaatgtgccttaacatctaagttgaggcc

SEQ ID NO: 27 - DNA, 85 bp, Artificial Sequence
  PCR primer
ccccctttttaggcctccccgtttatcctcccaaaatgtaaaatataattaaaatatattaataaacttcgattaataaacttcg

SEQ ID NO: 28 - DNA, 43 bp, Artificial Sequence
  PCR primer
aaaaggatccccttttagcgtataaagttttatatagctattg

SEQ ID NO: 29 - DNA, 33 bp, Artificial Sequence
  PCR primer
catgttctattgttactatggaagaggtagtag

SEQ ID NO: 30 - DNA, 29 bp, Artificial Sequence
  PCR primer
gcagttattataaatgctgctactagagc

(19) United States
(12) Patent Application Publication
(10) Pub. No.: US 2010/0086982 A1
(43) Pub. Date: Apr. 8, 2010

(54) PROCESS FOR THE BIOLOGICAL PRODUCTION OF n-BUTANOL WITH HIGH YIELD

(75) Inventor: Philippe Soucaille, Deyme (FR)

Correspondence Address: Pepper Hamilton LLP, 400 Berwyn Park, 899 Cassatt Road, Berwyn, PA, 19312-1183, US

(73) Assignee: METABOLIC EXPLORER, Saint Beauzire (FR)

(21) Appl. No.: 12/447,726

(22) PCT Filed: Oct. 29, 2007
(86) PCT No.: PCT/EP07/61634

§ 371 Date: Jun. 11, 2009

(30) Foreign Application Priority Data
Oct. 31, 2006 (EP) PCT/EP2006/067993

(51) Int. Cl. C12P 7/16; C12N 1/00; C12N 1/21

(52) U.S. Cl. 435/160; 435/243; 435/252.3

(57) ABSTRACT

The present invention provides a method for the biological production of n-butanol at high yield from a fermentable carbon source. In one aspect of the present invention, a process for the conversion of glucose to n-butanol is achieved by the use of a recombinant organism comprising a host C. acetobutilicum transformed i) to eliminate the butyrate pathway ii) to eliminate the acetone pathway iii) to eliminate the lactate pathway and iv) to eliminate the acetate pathway. In another aspect of the present invention, the hydrogen flux is decreased and the reducing power redirected to n-butanol production by attenuating the expression of the hydrogenase gene. Optionally the n-butanol produced can be eliminated during the fermentation by gas striping and further purified by distillation.

### FIELD OF INVENTION

The invention comprises a process for the bioconversion of a fermentable carbon source to n-butanol at high yield by a metabolically engineered microorganism.

### BACKGROUND OF THE INVENTION

n-Butanol is a colorless, neutral liquid of medium volatility with restricted miscibility (about 7-8%) in water, but freely miscible with all common solvents such as glycols, ketones, alcohol, aldehydes, ethers, and aromatic and aliphatic hydrocarbons. n-Butanol is used i) to make other chemicals, ii) as a solvent and iii) as an ingredient in formulated products such as cosmetics. The major uses of n-butanol as a feed-stock are in the synthesis of acrylate/methacrylate esters, glycol ethers, n-Butyl acetate, amino resins and n-Butylamines. Currently more than 9 millions tons of n-Butanol are consumed annually in the world.

More recently it has been shown that n-butanol is a better bio fuel than ethanol due to lower vapour pressure, higher energy content (closer to that of gasoline) and lesser susceptibility to separation in the presence of water. Furthermore, n-butanol can be blended at higher concentrations than ethanol for use in standard vehicle engines and it does not require automakers to compromise on performance to meet environmental regulations; it is also suitable for transport in pipelines and as a result it has the potential to be introduced into gasoline quickly and avoid the need for additional large-scale supply infrastructures.

n-butanol can be produced as an acetone/n-butanol/ethanol (ABE) mixture by the fermentation of carbohydrate by solventogenic Clostridia. The ABE fermentations are biphasic. During the first acidogenic phase, high growth rate is accompanied by acetic and butyric acids production. In the second solventogenic phase growth rate decrease and the solvents (ABE) are produced with the concomitant consumption of the organic acids produced in the first phase. Carbon dioxide and hydrogen are produced throughout the fermentation.

The biological production of n-butanol, presented in FIG. 1, requires the formation of butyryl-CoA as an intermediate which can be reduced, depending on the physiological conditions, by two different bi-functional aldehyde-alcohol dehydrogenases encoded by adhE1 and adhE2. Butyryl-CoA can also be converted to butyric acid by a phospho-transbutyrylase and a butyrate kinase encoded respectively by the ptb and buk genes. Acetone is produced from aceto-acetyl-CoA (an intermediate in the production of butyryl-CoA) by a CoA-transferase and an acetoacetate decarboxylase encoded respectively by the ctfAB and adc genes. Hydrogen is produced by an iron only hydrogenase encoded by the hydA gene. When cultures are performed in the presence of carbon monoxide, a hydrogenase inhibitor, n-butanol, ethanol and lactate are the main fermentation products. Lactate is produced from pyruvate by a lactate dehydrogenase encoded by the ldh gene.

Clostridium acetobutylicum strains with an inactivated buk gene (obtained by single crossing over with a non-replicable plasmid) have already been described in the article (Green et al., 1996). The non-replicable vector pJC4BK, with a 0.8 kb internal buk fragment was integrated into the chromosomal buk gene which leaded to an inactivation of the endogenous gene. The obtained strain was named “mutant PJC4BK” from the name of the plasmid. As precised in this article, this gene integration did not completely eliminate enzyme activity nor butyrate formation due to the instability of this type of gene inactivation that can reverse to wild type by plasmid excision. This mutant strain was then used in several studies (Green and Bennett, 1998; Desai and Harris, 1999; Harris et al., 2000).

Traditionally, the commercial ABE fermentation was conducted only in a batch mode due to continuous cultures instability of the producing Clostridia. Several solvent yielding fermentation processes have been described. These processes yield n-butanol, acetone and ethanol in a ratio of 6:3:1. Solvent yields of 29-34% (18-25% for n-butanol only) of fermentable carbon source have been reported in the literature. A total solvent concentration of 16-24 g/l and a n-butanol concentration of 10-14 g/l is generally the limit due to toxicity of n-butanol produced. However, these low titers of solvent no longer seem to be an economical limitation to the process as it has recently been demonstrated that solvents can be recovered during fermentation by the use of the “low cost” gas striping technology.

The problem to be solved by the present invention is to obtain a stable mutant strain with no butyrate kinase activity, that could be cultureD for several generations without any possibility of reversion to the wild type genotype. This strain would be useful for the biological production of n-butanol at high yield, from an inexpensive carbon substrate such as glucose or other sugars, by genetically stable cultures of Clostridia. The number of biochemical steps to inactivate and the complexity of the regulation of the metabolism necessitate, for an industrial feasible process of n-butanol production, the use of a metabolically engineered whole cell catalyst.

### SUMMARY OF THE INVENTION

Applicants have solved the stated problem and the present invention provides a method for bioconverting a fermentable carbon source to n-butanol as a major product by genetically stable cultures of Clostridia. Glucose is used as a model substrate and recombinant Clostridium acetobutylicum is used as the model host. In one aspect of this invention, a stable recombinant C. acetobutylicum unable to metabolize butyryl-CoA to butyrate is constructed by deleting the gene coding for the butyrate kinase (buk). In another aspect of this invention, a recombinant C. acetobutylicum unable to produce acetone is constructed by deleting the genes coding for the CoA-transferase (ctfAB). In a further aspect of this invention a recombinant strain unable to produce lactate is constructed by deleting the gene coding for the lactate dehydrogenase (ldh). Furthermore, a recombinant C. acetobutylicum unable to produce acetate is constructed by deleting the genes coding for the phosphotransacetylase and/or acetate kinase (pta and ack). In a final aspect of this invention, the flux of hydrogen production is decreased and then the flux of reducing equivalent redirected toward n-butanol production by attenuating the gene encoding the hydrogenase (hydA).

The present invention may be generally applied to include any carbon substrate that is readily converted to acetyl-coA.

Accordingly it is an object of the present invention to provide a recombinant organism, useful for the production of n-butanol comprising: (a) at least deletion of one of the two genes involved in the conversion of butyryl-CoA to butyrate and (b) at least deletion of one of the two genes encoding the CoA-transferase activity. Optionally the recombinant organism may comprise i) inactivating mutations in endogenous genes selected from the group consisting of: (a) a gene encoding a polypeptide having lactate dehydrogenase activity (b) a gene encoding a polypeptide having phospho-transacetylase or actate kinase activity and ii) attenuation in a gene encoding a polypeptide having hydrogenase activity.

In another embodiment the invention provides a stable process for the production of n-butanol at high yield from a recombinant organism comprising: (a) contacting the recombinant organism of the present invention with at least one carbon source selected from the group consisting of monosaccharides, oligosaccharides, polysaccharides, and single-carbon substrates whereby n-butanol is produced; optionally (b) recovering the n-butanol during the production through a step of gas striping and (c) purifying n-butanol from the condensate by distillation.

### BRIEF DESCRIPTION OF THE DRAWINGS

The accompanying drawing which is incorporated in and constitutes a part of this specification exemplifies the invention and together with the description, serve to explain the principles of this invention.

FIG. 1 depicts the genetic engineering of central metabolism in the development of a butanol production system from carbohydrates.

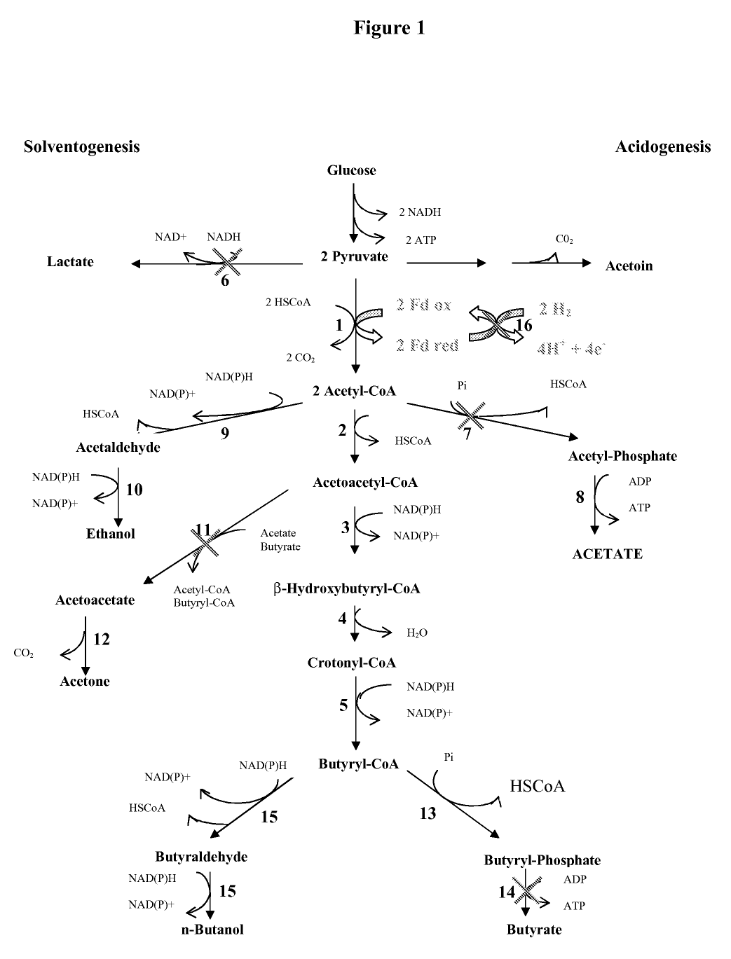

1: Pyruvate-ferredoxin oxydoreductase; 2: Thiolase; 3: β-Hydroxybutyryl-CoA dehydrogenase; 4: Crotonase; 5: Butyryl-CoA dehydrogenase; 6: Lactate dehydrogenase; 7: Phospho-transacetylase; 8: Acetate kinase; 9: Acetaldehyde deshydrogenase; 10: Ethanol dehydrogenase; 11: CoA transferase (Acetoacetyl-CoA:acetate/butyrate:CoA transferase); 12: Acetoacetate decarboxylase; 13: Phospho-transbutyrylase; 14: Butyrate kinase; 15: Butyraldehyde-Butanol dehydrogenase; 16: hydrogenase.

### DETAILED DESCRIPTION OF THE INVENTION

As used herein the following terms may be used for interpretation of the claims and specification.

The term “microorganism” refers to all kind of unicellular organisms, including prokaryotic organisms like bacteria, and eukaryotic organisms like yeasts.

The expression “appropriate culture medium” refers to a culture medium adapted for the used microorganism as it is well known by the man skilled in the art.

The term “carbon substrate” or “source of carbon” means any carbon source capable of being metabolized by a microorganism wherein the substrate contains at least one carbon atom. Authors refer particularly to renewable, inexpensive and fermentable carbon sources such as monosaccharides, oligosaccharides, polysaccharides, single-carbon substrates, and polyols such as glycerol. Single carbon substrate are defined as carbon molecules that contain only one carbon atom such as methanol.

Monosaccharides of the formula (CH₂O) are also called oses or “simple sugars”; monosaccharides include saccharose, fructose, glucose, galactose and mannose.

Other carbon sources comprising more than one monosaccharide are called disaccharides, trisaccharides, oligosaccharides and polysaccharides. Disaccharides include saccharose (sucrose), lactose and maltose. Starch and hemicellulose are polysaccharides, also known as “complex sugars”.

Therefore, the term “source of carbon” means any product cited above, and mixture thereof.

The term “attenuation” refers to a decreased expression of a gene or a decreased activity of the protein, product of the gene. The man skilled in the art knows numerous means to obtain this result, and for example:
Introduction of a mutation into the gene, decreasing the expression level of this gene, or the level of activity of the encoded protein.
Replacement of the natural promoter of the gene by a low strength promoter, resulting in a lower expression
Use of elements destabilizing the corresponding messenger RNA or the protein
Deletion of the gene if no expression is needed.

The term “deleted gene” means that a substantial part of the coding sequences of said gene was removed. Preferably, at least 50% of the coding sequence was removed, and more preferably at least 80%.

In the description of the present invention, enzymes are identified by their specific activities. This definition thus includes all polypeptides that have the defined specific activity also present in other organisms, more particularly in other microorganisms. Often enzymes with similar activities can be identified by their grouping to certain families defined as PFAM or COG.

PFAM (protein families database of alignments and hidden Markov models; http://www.sanger.ac.uk/Software/Pfam) represents a large collection of protein sequence alignments. Each PFAM makes it possible to visualize multiple alignments, see protein domains, evaluate distribution among organisms, gain access to other databases, and visualize known protein structures.

COGs (clusters of orthologous groups of proteins; http://www.ncbi.nlm.nih.gov/COG) are obtained by comparing protein sequences from 43 fully sequenced genomes representing 30 major phylogenic lines. Each COG is defined from at least three lines, which permits the identification of former conserved domains.

The means of identifying homologous sequences and their percentage homologies are well known to those skilled in the art, and include in particular the BLAST programs, which can be used from the website http://www.ncbi.nlm.nih.gov/BLAST/ with the default parameters indicated on that website. The sequences obtained can then be exploited (e.g., aligned) using, for example, the programs CLUSTALW (http://www.ebi.ac.uk/clustalw/) or MULTALIN (http://prodes.toulouse.inra.fr/multalin/cgi-bin/multalin.pl), with the default parameters indicated on those websites.

Using the references given on GenBank for known genes, those skilled in the art are able to determine the equivalent genes in other organisms, bacterial strains, yeasts, fungi, mammals, plants, etc. This routine work is advantageously done using consensus sequences that can be determined by carrying out sequence alignments with genes derived from other microorganisms, and designing degenerate probes to clone the corresponding gene in another organism. These routine methods of molecular biology are well known to those skilled in the art, and are described, for example, in Sambrook et al. (Molecular Cloning: a Laboratory Manual. 2^{nd} ed. Cold Spring Harbor Lab., Cold Spring Harbor, N.Y., 1989.).

The present invention provides a method for the fermentative batch or continuous production of n-butanol by culturing a microorganism in an appropriate culture medium comprising a carbon source and the simultaneous recovery of n-butanol from the culture medium wherein at least one gene involved in butyrate formation is deleted in the microorganism.

A specific embodiment of the invention provides a method wherein the microorganism is modified to be unable to convert butyryl-CoA to butyrate due to the deletion of at least one gene encoding for phospho-transbutyrylase (ptb) or butyrate kinase (buk). Deletion of genes in Clostridia can be done using the method recently described in patent application PCT/EP2006/066997 allowing the i) replacement of the gene to delete with an erythromycin resistance gene and ii) removal of the erythromycin resistance gene with a recombinase.

In another embodiment of the invention, the microorganism is unable to produce acetone due to an attenuation or a deletion of at least one of the gene encoding for CoA-transferase (ctfAB) or acetoacetate decarboxylase (adc). Deletion of one of these genes can be done using the method recently described in patent application PCT/EP2006/066997.

In a further embodiment of the invention, the microorganism used in the method of the invention is unable to produce lactate. In particular this can be due to a deletion of the gene ldh encoding for lactate dehydrogenase. Deletion of ldh can be done using the method recently described in patent application PCT/EP2006/066997.

In another embodiment, the microorganism is modified in such a way to be unable to produce acetate. This result can be achieved by deletion of at least one of the genes encoding for phospho-transacetylase (pta) or acetate kinase (ack). Deletion of one of these genes can be done using the method recently described in patent application PCT/EP2006/066997.

An embodiment of the invention also provides a microorganism with a decreased flux of hydrogen production and then a redirection of the flux of reducing equivalent toward n-butanol production; this can be done by attenuating the gene encoding the hydrogenase (hydA), an enzyme that provides a sink for reducing equivalent in the form of hydrogen production. Attenuation of hydA can be done by replacing the natural promoter by a low strength promoter or by element destabilizing the corresponding messenger RNA or the protein. If needed, complete attenuation of the gene can also be achieved by a deletion of the corresponding DNA sequence.

Preferably, the used microorganism is selected among the group consisting of C. acetobutylicum, C. beijerinckii, C. saccharoperbutylacetonicum or C. saccharobutylicum.

In another embodiment of the invention, the culture is continuous and stable.

In another embodiment, the method according to the invention comprises the following steps:
(a) contacting a microorganism with at least one carbon source selected from the group consisting of glucose, xylose, arabinose, sucrose, monosaccharides, oligosaccharides, polysaccharides, cellulose, xylan, starch or its derivatives and glycerol, whereby n-butanol is produced
(b) Recovering the n-butanol during the fermentation by gas striping and
(c) Isolation of n-butanol from the condensate by distillation.

Those skilled in the art are able to define the culture conditions for the microorganisms according to the invention. In particular the clostridia are fermented at a temperature between 20° C. and 55° C., preferentially between 25° C. and 40° C., and more specifically about 35° C. for C. acetobutylicum.

The fermentation is generally conducted in fermentors with an inorganic culture medium of known defined composition adapted to the bacteria used, containing at least one simple carbon source, and if necessary a co-substrate necessary for the production of the metabolite.

The invention is also related to the microorganism as described previously. Preferably, this microorganism is selected among the group consisting of C. acetobutylicum, C. beijerinckii, C. saccharoperbutylacetonicum or C. saccharobutylicum.

### Example 1

### Construction of Strains Unable to Produce Butyrate

### Clostridium Acetobutylicum Δcac1515 Δupp Δbuk

To delete the buk gene, the homologous recombination strategy described by Croux & Soucaille (2006) in patent application PCT/EP2006/066997 is used. This strategy allows the insertion of an erythromycin resistance cassette, while deleting most of the gene concerned. The buk deletion cassette in pCons::upp was constructed as follows.

TABLE 1
primers sequences

| Name | | Primer sequences |
|---|---|---|
| Buk 1 | SEQ ID N° 1 | aaaaggatcctagtaaaagggagtgtacg |
| | | accagtg |
| Buk 2 | SEQ ID N° 2 | ggggtcgcgaaaaaaggggggattattag |
| | | taatctatacatgttaacattcctccac |
| Buk 3 | SEQ ID N° 3 | cccccttttttcgcgaccccacttcttgc |
| | | acttgcagaaggtggac |
| Buk 4 | SEQ ID N° 4 | aaaaggatcctctaaattctgcaatatat |
| | | gccccccc |
| Buk 0 | SEQ ID N° 5 | ataacaggatatatgctctctgacgcgg |
| Buk 5 | SEQ ID N° 6 | gatcatcactcattttaaacatggggcc |

Two DNA fragments surrounding buk were PCR amplified with the Pwo polymerase with total DNA from C. acetobutylicum as template and two specific couples of olignonucleotides. With the couples of primers BUK 1-BUK 2 and BUK 3-BUK 4, two DNA fragments were respectively obtained. Both primers BUK 1 and BUK 4 introduce a BamHI site while primers BUK 2 and BUK 3 have a complementary region which introduces a NruI site. DNA fragments BUK 1-BUK 2 and BUK 3-BUK 4 were joined in a PCR fusion experiment with primers BUK 1 and BUK 4 and the resulting fragment was cloned in pCR4-TOPO-Blunt to yield pTOPO:buk. At the unique StuI site of pTOPO:buk, an antibiotic resistance MLS gene with FRT sequences on both sides was introduced from the StuI fragment of pUC18-FRT-MLS2. The BUK deletion cassette obtained after BamHI digestion of the resulting plasmid was cloned into pCons::upp at the BamHI site to yield the pREPABUK::upp plasmid.

The pREPABUK::upp plasmid was used to transform by electroporation C. acetobutylicum MGCΔcac15Δupp strain. After selection on Petri plate for clones resistant to erythromycin (40 μg/ml), one colony was cultured for 24 hours in liquid synthetic medium with erythromycin at 40 μg/ml and 100 μl of undiluted culture was plated on RCA with erythromycin at 40 μg/ml and 5-FU at 400 μM. Colonies resistant to both erythromycin and 5-FU were replica plated on both RCA with erythromycin at 40 μg/ml and RCA with thiamphenicol at 50 μg/ml to select clones where 5-FU resistance is also associated with thiamphenicol sensitivity. The genotype of clones resistant to erythromycin and sensitive to thiamphenicol was checked by PCR analysis (with primers BUK 0 and BUK 5 located outside of the buk deletion cassette). The Δcac15ΔuppΔbuk::mls^{R} strain which have lost pREPΔbuk::upp was isolated.

The Δcac15ΔuppΔbuk::mls^{R} strain was transformed with pCLF1.1 vector expressing the Flp1 gene encoding the Flp recombinase from S. cerevisiae. After transformation and selection for resistance to thiamphenicol (50 μg/ml) on Petri plate, one colony was cultured on synthetic liquid medium with thiamphenicol at 50 μg/ml and appropriate dilutions were plated on RCA with thiamphenicol at 50 μg/ml. Thiamphenicol resistant clones were replica plated on both RCA with erythromycin at 40 μg/ml and RCA with thiamphenicol at 50 μg/ml. The genotype of clones with erythromycin sensitivity and thiamphenicol resistance was checked by PCR analysis with primers BUK 0 and BUK 5. Two successive 24 hours cultures of the Δcac15ΔuppΔbuk strain with erythromycin sensitivity and thiamphenicol resistance were carried out in order to lose pCLF1.1. The Δcac15ΔuppΔbuk strain which has lost pCLF1.1 was isolated according to its sensitivity to both erythromycin and thiamphenicol.

### Example 2

### Construction of Strains Unable to Produce Butyrate and Acetone

### C. Acetobutylicum Δcac1515 Δupp Δbuk ΔctfAB

To delete the ctfAB genes, the homologous recombination strategy described by Croux & Soucaille (2006) in patent application PCT/EP2006/066997 is used. This strategy allows the insertion of an erythromycin resistance cassette, while deleting most of the genes concerned. The ctfAB deletion cassette in pCons::upp was constructed as follows.

TABLE 2
primers sequences

| Name | | Primer sequences |
|---|---|---|
| Ctf 1 | SEQ ID N° 7 | aaaaggatcccagacactataatagcttta |
| | | ggtggtacccc |
| Ctf 2 | SEQ ID N° 8 | ggggaggcctaaaaagggggattataaaaa |
| | | gtagttgaaatatgaaggtttaaggttg |
| Ctf 3 | SEQ ID N° 9 | ccccctttttaggcctccccatatccaatg |
| | | aacttagacccatggctg |
| Ctf 4 | SEQ ID N° 10 | aaaaggatccgtgttataatgtaaatataa |
| | | ataaataggactagaggcg |
| Ctf 0 | SEQ ID N° 11 | taccaccttctttcacgcttggctgcgg |
| Ctf 5 | SEQ ID N° 12 | tatttaaagaggcattatcaccagagcg |

Two DNA fragments surrounding ctfAB were PCR amplified with the Pwo polymerase with total DNA from C. acetobutylicum as template and two specific couples of olignonucleotides. With the couples of primers CTF 1-CTF 2 and CTF 3-CTF 4, two DNA fragments were respectively obtained. Both primers CTF 1 and CTF 4 introduce a BamHI site while primers CTF 2 and CTF 3 have a complementary region which introduces a StuI site. DNA fragments CTF 1-CTF 2 and CTF 3-CTF 4 were joined in a PCR fusion experiment with primers CTF 1 and CTF 4 and the resulting fragment was cloned in pCR4-TOPO-Blunt to yield pTOPO:CTF. At the unique StuI site of pTOPO:CTF, an antibiotic resistance MLS gene with FRT sequences on both sides was introduced from the StuI fragment of pUC18-FRT-MLS2. The UPP deletion cassette obtained after BamHI digestion of the resulting plasmid was cloned into pCons::upp at the BamHI site to yield the pREPACTF::upp plasmid.

The pREPACTF::upp plasmid was used to transform by electroporation C. acetobutylicum MGCΔcac15ΔuppΔbuk strain. After selection on Petri plate for clones resistant to erythromycin (40 μg/ml), one colony was cultured for 24 hours in liquid synthetic medium with erythromycin at 40 μg/ml and 100 μl of undiluted culture was plated on RCA with erythromycin at 40 μg/ml and 5-FU at 400 μM. Colonies resistant to both erythromycin and 5-FU were replica plated on both RCA with erythromycin at 40 μg/ml and RCA with thiamphenicol at 50 μg/ml to select clones where 5-FU resistance is also associated with thiamphenicol sensitivity. The genotype of clones resistant to erythromycin and sensitive to thiamphenicol was checked by PCR analysis (with primers CTF 0 and CTF 5 located outside of the ctfAB deletion cassette). The Δcac15ΔuppΔbuk ΔctfAB::mls^{R} strain which have lost pREPACTF::upp was isolated.

The Δcac15ΔuppΔbukΔctfAB::mls^{R} strain was transformed with pCLF1.1 vector expressing the Flp1 gene encoding the Flp recombinase from S. cerevisiae. After transformation and selection for resistance to thiamphenicol (50 μg/ml) on Petri plate, one colony was cultured on synthetic liquid medium with thiamphenicol at 50 μg/ml and appropriate dilutions were plated on RCA with thiamphenicol at 50 μg/ml. Thiamphenicol resistant clones were replica plated on both RCA with erythromycin at 40 μg/ml and RCA with thiamphenicol at 50 μg/ml. The genotype of clones with erythromycin sensitivity and thiamphenicol resistance was checked by PCR analysis with primers CTF 0 and CTF 5. Two successive 24 hours cultures of the Δcac15ΔuppΔbukΔctfAB strain with erythromycin sensitivity and thiamphenicol resistance were carried out in order to lose pCLF1.1. The Δcac15ΔuppΔbukΔctfAB strain which has lost pCLF1.1 was isolated according to its sensitivity to both erythromycin and thiamphenicol.

### Example 3

### Construction of Strains Unable to Produce Butyrate, Acetone and Lactate

### C. Acetobutylicum Δcac1515 Δupp Δbuk ΔctfAB Δldh

To delete the ldh gene, the homologous recombination strategy described by Croux & Soucaille (2006) in patent application PCT/EP2006/066997 is used. This strategy allows the insertion of an erythromycin resistance cassette, while deleting most of the genes concerned. The ldh deletion cassette in pCons::upp was constructed as follows.

TABLE 3
primers sequences

| Name | | Primer sequences |
|---|---|---|
| Ldh 1 | SEQ ID N° 13 | AAAAGGATCCGCTTTAAAATTTGGAAAG |
| | | AGGAAGTTGTG |
| Ldh 2 | SEQ ID N° 14 | GGGGAGGCCTAAAAAGGGGGTTAGAAAT |
| | | CTTTAAAAATTTCTCTATAGAGCCCATC |
| Ldh 3 | SEQ ID N° 15 | CCCCCTTTTTAGGCCTCCCCGGTAAAAG |
| | | ACCTAAACTCCAAGGGTGGAGGCTAGGT |
| | | C |
| Ldh 4 | SEQ ID N° 16 | AAAAGGATCCCCCATTGTGGAGAATATT |
| | | CCAAAGAAGAAAATAATTGC |
| Ldh 0 | SEQ ID N° 17 | CAGAAGGCAAGAATGTATTAAGCGGAAA |
| | | TGC |
| Ldh 5 | SEQ ID N° 18 | CTTCCCATTATAGCTCTTATTCACATTA |
| | | AGC |

Two DNA fragments surrounding ldh (CAC267) were PCR amplified with the Pwo polymerase with total DNA from C. acetobutylicum as template and two specific couples of olignonucleotides. With the couples of primers LDH 1-LDH 2 and LDH 3-LDH 4, 1135 by and 1177 by DNA fragments were respectively obtained. Both primers LDH 1 and LDH 4 introduce a BamHI site while primers LDH 2 and LDH 3 have a complementary region which introduces a StuI site. DNA fragments LDH 1-LDH 2 and LDH 3-LDH 4 were joined in a PCR fusion experiment with primers LDH 1 and LDH 4 and the resulting fragment was cloned in pCR4-TOPO-Blunt to yield pTOPO:LDH. At the unique StuI site of pTOPO:LDH, an antibiotic resistance MLS gene with FRT sequences on both sides was introduced from the 1372 by StuI fragment of pUC18-FRT-MLS2. The UPP deletion cassette obtained after BamHI digestion of the resulting plasmid was cloned into pCons::upp at the BamHI site to yield the pREPΔLDH::upp plasmid.

The pREPΔLDH::upp plasmid was used to transform by electroporation C. acetobutylicum MGCΔcac15ΔuppΔbukΔctfAB strain. After selection on Petri plate for clones resistant to erythromycin (40 μg/ml), one colony was cultured for 24 hours in liquid synthetic medium with erythromycin at 40 μg/ml and 100 μl of undiluted culture was plated on RCA with erythromycin at 40 μg/ml and 5-FU at 400 μM. Colonies resistant to both erythromycin and 5-FU were replica plated on both RCA with erythromycin at 40 μg/ml and RCA with thiamphenicol at 50 μg/ml to select clones where 5-FU resistance is also associated with thiamphenicol sensitivity. The genotype of clones resistant to erythromycin and sensitive to thiamphenicol was checked by PCR analysis (with primers LDH 0 and LDH 5 located outside of the ldh deletion cassette). The Δcac15ΔuppΔbuk ΔctfAB Δldh::mls^{R} strain which have lost pREPΔLDH::upp was isolated.

The Δcac15ΔuppΔbukΔctfABΔldh::mls^{R} strain was transformed with pCLF1.1 vector expressing the Flp1 gene encoding the Flp recombinase from S. cerevisiae. After transformation and selection for resistance to thiamphenicol (50 μg/ml) on Petri plate, one colony was cultured on synthetic liquid medium with thiamphenicol at 50 μg/ml and appropriate dilutions were plated on RCA with thiamphenicol at 50 μg/ml. Thiamphenicol resistant clones were replica plated on both RCA with erythromycin at 40 μg/ml and RCA with thiamphenicol at 50 μg/ml. The genotype of clones with erythromycin sensitivity and thiamphenicol resistance was checked by PCR analysis with primers LDH 0 and LDH 5. Two successive 24 hours cultures of the Δcac15ΔuppΔbukΔctfAB Δldh strain with erythromycin sensitivity and thiamphenicol resistance were carried out in order to lose pCLF1.1. The Δcac15ΔuppΔbukΔctfABΔldh strain which has lost pCLF1.1 was isolated according to its sensitivity to both erythromycin and thiamphenicol.

### Example 4

### Construction of Strains Unable to Produce Butyrate, Acetone, Lactate and Acetate

### C. Acetobutylicum Δcac1515 Δupp Δbuk ΔctfAB Δldh Δpta-ack

To delete the pta and ack genes, the homologous recombination strategy described by Croux & Soucaille (2006) in patent application PCT/EP2006/066997 is used. This strategy allows the insertion of an erythromycin resistance cassette, while deleting most of the genes concerned. The pta-ack deletion cassette in pCons::upp was constructed as follows.

TABLE 4
primers sequences

| Name | | Primer sequences |
|---|---|---|
| PA 1 | SEQ ID N° 19 | aaaaggatcctattataacagtcaaacccaa |
| | | taaaatactggg |
| PA 2 | SEQ ID N° 20 | ggggaggcctaaaaagggggttaatccattt |
| | | gtattttctcccttcataatgcc |
| PA 3 | SEQ ID N° 21 | ccccctttttaggcctcccctttattttgca |
| | | tgcttatataataaattatggctgcg |
| PA 4 | SEQ ID N° 22 | aaaaggatccgcttttccttcttttacaaga |
| | | tttaaagcc |
| PA 0 | SEQ ID N° 23 | cacttttatttatcaagctgtaggcc |
| PA 5 | SEQ ID N° 24 | tataccttttgaacctaggaaaggc |

Two DNA fragments surrounding pta-ack were PCR amplified with the Pwo polymerase with total DNA from C. acetobutylicum as template and two specific couples of olignonucleotides. With the couples of primers PA 1-PA 2 and PA 3-PA 4, two DNA fragments were respectively obtained. Both primers PA 1 and PA 4 introduce a BamHI site while primers PA 2 and PA 3 have a complementary region which introduces a StuI site. DNA fragments PA 1-PA 2 and PA 3-PA 4 were joined in a PCR fusion experiment with primers PA 1 and PA 4 and the resulting fragment was cloned in pCR4-TOPO-Blunt to yield pTOPO:PA. At the unique StuI site of pTOPO:PA, an antibiotic resistance MLS gene with FRT sequences on both sides was introduced from the StuI fragment of pUC18-FRT-MLS2. The UPP deletion cassette obtained after BamHI digestion of the resulting plasmid was cloned into pCons::upp at the BamHI site to yield the pREPΔPA::upp plasmid. The pREPΔPA::upp plasmid was used to transform by electroporation C. acetobutylicum MGCΔcac15ΔuppΔbukΔctfABΔldh strain. After selection on Petri plate for clones resistant to erythromycin (40 μg/ml), one colony was cultured for 24 hours in liquid synthetic medium with erythromycin at 40 μg/ml and 100 μl of undiluted culture was plated on RCA with erythromycin at 40 μg/ml and 5-FU at 400 μM. Colonies resistant to both erythromycin and 5-FU were replica plated on both RCA with erythromycin at 40 μg/ml and RCA with thiamphenicol at 50 μg/ml to select clones where 5-FU resistance is also associated with thiamphenicol sensitivity. The genotype of clones resistant to erythromycin and sensitive to thiamphenicol was checked by PCR analysis (with primers PA 0 and PA 5 located outside of the pta-ack deletion cassette). The Δcac15ΔuppΔbuk ΔctfABΔldhΔpta-ack::mls^{R} strain which have lost pREPΔPA::upp was isolated.

The Δcac15ΔuppΔbukΔctfABΔldh Δpta-ack::mls^{R} strain was transformed with pCLF1.1 vector expressing the Flp1 gene encoding the Flp recombinase from S. cerevisiae. After transformation and selection for resistance to thiamphenicol (50 μg/ml) on Petri plate, one colony was cultured on synthetic liquid medium with thiamphenicol at 50 μg/ml and appropriate dilutions were plated on RCA with thiamphenicol at 50 μg/ml. Thiamphenicol resistant clones were replica plated on both RCA with erythromycin at 40 μg/ml and RCA with thiamphenicol at 50 μg/ml. The genotype of clones with erythromycin sensitivity and thiamphenicol resistance was checked by PCR analysis with primers PA 0 and PA 5. Two successive 24 hours cultures of the Δcac15ΔuppΔbukΔctfABΔldhΔpta-ack strain with erythromycin sensitivity and thiamphenicol resistance were carried out in order to lose pCLF1.1. The Δcac15ΔuppΔbukΔctfABΔldhΔpta-ack strain which has lost pCLF1.1 was isolated according to its sensitivity to both erythromycin and thiamphenicol.

### Example 5

### Construction of Strains with Lower Hydrogen Production

### C. Acetobutylicum Δcac1515 Δupp Δbuk ΔctfAB Δldh ΔhydA

To delete the hydA gene, the homologous recombination strategy described by Croux & Soucaille (2006) in patent application PCT/EP2006/066997 is used. This strategy allows the insertion of an erythromycin resistance cassette, while deleting most of the genes concerned. The hydA deletion cassette in pCons::upp was constructed as follow.

TABLE 5
primers sequences

| Name | | Primer sequences |
|---|---|---|
| Hyd 1 | SEQ ID N° 25 | AAAAGGATCCGCCTCTTCTGTATTATGCAA |
| | | GGAAAGCAGCTGC |
| Hyd 2 | SEQ ID N° 26 | GGGGAGGCCTAAAAAGGGGGTATATAAAAT |
| | | AAATGTGCCTTAACATC |
| | | TAAGTTGAGGCC |
| Hyd 3 | SEQ ID N° 27 | CCCCCTTTTTAGGCCTCCCCGTTTATCCTC |
| | | CCAAAATGTAAAATATAA |
| | | TTAAAATATATTAATAAACTTCGATTAATA |
| | | AACTTCG |
| Hyd 4 | SEQ ID N° 28 | AAAAGGATCCCCTTTTAGCGTATAAAGTTT |
| | | TATATAGCTATTG |
| Hyd 0 | SEQ ID N° 29 | CATGTTCTATTGTTACTATGGAAGAGGTA |
| | | GTAG |
| Hyd 5 | SEQ ID N° 30 | GCAGTTATTATAAATGCTGCTACTAGAGC |

Two DNA fragments surrounding hydA (CAC028) were PCR amplified with the Pwo polymerase with total DNA from C. acetobutylicum as template and two specific couples of olignonucleotides. With the couples of primers HYD 1-HYD 2 and HYD 3-HYD 4, 1269 bp and 1317 by DNA fragments were respectively obtained. Both primers HYD 1 and HYD 4 introduce a BamHI site while primers HYD 2 and HYD 3 have a complementary region which introduces a StuI site. DNA fragments HYD 1-HYD 2 and HYD 3-HYD 4 were joined in a PCR fusion experiment with primers HYD 1 and HYD 4 and the resulting fragment was cloned in pCR4-TOPO-Blunt to yield pTOPO:HYD. At the unique StuI site of pTOPO:HYD, an antibiotic resistance MLS gene with FRT sequences on both sides was introduced from the 1372 by StuI fragment of pUC18-FRT-MLS2. The UPP deletion cassette obtained after BamHI digestion of the resulting plasmid was cloned into pCons::upp at the BamHI site to yield the pREPΔHYD::upp plasmid.

The pREPΔHYD::upp plasmid was used to transform by electroporation C. acetobutylicum MGCΔcac15ΔuppΔbukΔctfABΔldh strain. After selection on Petri plate for clones resistant to erythromycin (40 μg/ml), one colony was cultured for 24 hours in liquid synthetic medium with erythromycin at 40 μg/ml and 100 μl of undiluted culture was plated on RCA with erythromycin at 40 μg/ml and 5-FU at 400 μM. Colonies resistant to both erythromycin and 5-FU were replica plated on both RCA with erythromycin at 40 μg/ml and RCA with thiamphenicol at 50 μg/ml to select clones where 5-FU resistance is also associated with thiamphenicol sensitivity. The genotype of clones resistant to erythromycin and sensitive to thiamphenicol was checked by PCR analysis (with primers HYD 0 and HYD 5 located outside of the hydA deletion cassette). The Δcac15ΔuppΔbukΔctfABΔldhΔhydA::mls^{R} strain which have lost pREPΔHYD::upp was isolated.

The Δcac15ΔuppΔbukΔctfABΔldhΔhydA::mls^{R} strain was transformed with pCLF1.1 vector expressing the Flp1 gene encoding the Flp recombinase from S. cerevisiae. After transformation and selection for resistance to thiamphenicol (50 μg/ml) on Petri plate, one colony was cultured on synthetic liquid medium with thiamphenicol at 50 μg/ml and appropriate dilutions were plated on RCA with thiamphenicol at 50 μg/ml. Thiamphenicol resistant clones were replica plated on both RCA with erythromycin at 40 μg/ml and RCA with thiamphenicol at 50 μg/ml. The genotype of clones with erythromycin sensitivity and thiamphenicol resistance was checked by PCR analysis with primers HYD 0 and HYD 5. Two successive 24 hours cultures of the Δcac15ΔuppΔbukΔctfABΔldhΔhydA strain with erythromycin sensitivity and thiamphenicol resistance were carried out in order to lose pCLF1.1. The Δcac15ΔuppΔbukΔctfABΔldhΔhydA strain which has lost pCLF1.1 was isolated according to its sensitivity to both erythromycin and thiamphenicol.

### Example 6

### Batch Fermentation of N-Butanol Producing Strains

Strains were initially analyzed in anaerobic flask cultures in the synthetic medium described by Soni et al (Soni et al, 1987, Appl. Microbiol. Biotechnol. 27:1-5) supplemented with 2.5 g/l of ammonium acetate. An overnight culture at 35° C. was used to inoculate a 30 ml culture to an OD600 of 0.05. After incubation of the culture for 3 days at 35° C., glucose, organic acids and solvents were analyzed by HPLC using a Biorad HPX 97H column for the separation and a refractometer for the detection.

Strains with the correct phenotype were subsequently tested under production conditions in 300 ml fermentors (DASGIP) using an anaerobic batch protocol.

For this purpose the fermentor was filled with 250 ml of synthetic medium, sparged with nitrogen for 30 min and inoculated with 25 ml of preculture to an optical density (OD600 nm) between 0.05 and 0.1.

The temperature of the culture was maintained constant at 35° C. and the pH was permanently adjusted at 5.5 using an NH₄OH solution. The agitation rate was maintained at 300 rpm during the fermentation.

### Example 7

### Continuous Fermentation of N-Butanol Producing Strains

The best n-butanol producing strain was analyzed in chemostat cultures in the synthetic medium described by Soni et al (Soni et al, 1987, Appl. Microbiol. Biotechnol. 27:1-5). An overnight culture at 35° C. was used to inoculate a 300 ml fermentors (DASGIP) using an anaerobic chemostat protocol.

For this purpose the fermentor was filled with 250 ml of synthetic medium, sparged with nitrogen for 30 min and inoculated with 25 ml of preculture to an optical density (OD600 nm) between 0.05 and 0.1. After 12 hours of batch culture at 35° C., pH 5.5 (regulated using an NH₄OH solution) and an agitation rate of 300 rpm, the fermentor was continuously fed with oxygen free synthetic medium at a dilution rate of 0.05 h-1 while the volume was kept constant by sequential removal of fermentated medium. Stability of the culture was followed by products analysis using the HPLC protocol previously described.

### Example 8

### Evaluation of N-Butanol Production Strains in Batch Cultures

Production strains were evaluated in small flasks. 10% of thawed cultures (typically 3 ml) were used to inoculate 30 ml of synthetic medium (MSL4). A 15 minutes thermal shock at 80° C. was applied to kill any vegetative cells present before the initiation of growth. The cultures were then grown at 37° C. for 6 to 7 days. Extra-cellular compounds were quantified by HPLC using the following parameters: Eluent (H2SO4) concentration: 0.25 mM; Flow: 0.5 ml/min; Temperature: 25° C., Time: 50 minutes.

TABLE 6
Synthetic medium composition (MSL4).

| Compound | Concentration | |
|---|---|---|
| Glucose | 60 | g/l |
| KH2PO4 | 0.5 | g/l |
| K2HPO4 | 0.5 | g/l |
| MgSO4, 7H2O | 0.2 | g/l |
| FeSO4, 7H2O | 0.01 | g/l |
| CH3COOH | 2.2 | g/l |
| Aminobenzoic acid (p) | 8 | mg/l |
| Biotine | 0.04 | mg/l |

As can be seen in table 2, upon deletion of the gene coding for the butyrate kinase (buk) the maximum butyrate concentration detected in the medium decreases from 3.13 g/l after 2 days of culture in the C. acetobutylicum Δcac15 Δupp strain, to 0.43 g/l after 5 days of culture in the C. acetobutylicum Δcac15 Δupp Δbuk::MSLr strain. Notably, the alcohol/glucose yield (Y_{bu}+Yₑₜ) increases significantly, whereas the acetone/glucose yield (Y_{ac}) decreases significantly.

TABLE 7
Solvents yield in % g product/g glucose produced
and maximal butyrate concentration in g/l, in
batch culture by strains described above.

| | | | | | Butyrate MC | |
|---|---|---|---|---|---|---|
| Genotype | Y_{bu} + Yₑₜ | SD | Y_{ac} | SD | 48 H | 120 H |
| C. acetobutylicum | 23.47 | 0.01 | 9.94 | 0.01 | 3.13 | 1.61 |
| ATCC 824 Δcac15 | | | | | | |
| Δupp | | | | | | |
| C. acetobutylicum | 38.07 | 2.23 | 4.64 | 2.68 | 0.29 | 0.43 |
| ATCC 824 Δcac15 | | | | | | |
| Δupp Δbuk::MSLr | | | | | | |

SD denotes the standard deviation; MC denotes the Maximum Concentration in g/l.

### REFERENCES

Desai R P, Harris L M, Welker N E, Papoutsakis E T.
Metabolic flux analysis elucidates the importance of the acid-formation pathways in regulating solvent production by Clostridium acetobutylicum.
Metab Eng. 1999, 1:206-13.
Green E M, Bennett G N.
Genetic manipulation of acid and solvent formation in clostridium acetobutylicum ATCC 824
Biotechnol Bioeng. 1998, 58:215-21.
Green E M, Boynton Z L, Harris L M, Rudolph F B, Papoutsakis E T, Bennett G N.
Genetic manipulation of acid formation pathways by gene inactivation in Clostridium acetobutylicum ATCC 824.
Microbiology. 1996, 142:2079-86.
Harris L M, Desai R P, Welker N E, Papoutsakis E T.
Characterization of recombinant strains of the Clostridium acetobutylicum butyrate kinase inactivation mutant: need for new phenomenological models for solventogenesis and butanol inhibition?
Biotechnol Bioeng. 2000; 67:1-11.
Soni B. K., Soucaille P. Goma G.
Continuous acetone butanol fermentation: influence of vitamins on the metabolic activity of Clostridium acetobutylicum.
Appl. Microbiol. Biotechnol. 1987. 27: 1-5.

1. A method for the production of n-butanol by culturing a microorganism in an appropriate culture medium comprising a source of carbon and recovery of n-butanol from the culture medium, wherein at least one gene involved in butyrate formation is deleted in the microorganism.

2. The method according to claim 1 wherein the deleted gene is at least one of the following genes:
ptb encoding phospho-transbutyrylase and
buk encoding butyrate kinase.

3. The method according to claim 1 wherein at least one gene involved in acetone formation is attenuated in the microorganism.

4. The method according to claim 3 wherein at least one of the following genes is deleted:
ctfAB encoding CoA-transferase and
adc encoding aceto-acetate decarboxylase.

5. The method according to claim 1 wherein the microorganism is modified to be unable to produce lactate.

6. The method according to claim 5 wherein the ldh gene is deleted.

7. The method according to claim 1 wherein the microorganism is modified to be unable to produce acetate.

8. The method according to claim 7 wherein at least one gene involved in acetate formation is deleted.

9. method according to claim 8 wherein the deleted gene is
pta encoding phospho-transacetylase or
ack encoding acetate kinase.

10. The method according to claim 1 wherein the hydrogen flux is decreased and the reducing power redirected to butanol production.

11. The method according to claim 10 wherein the hydA gene is attenuated.

12. The method according to claim 1 wherein the microorganism is C. acetobutylicum, C. beijerinckii, C. saccharoperbutylacetonicum or C. saccharobutylicum.

13. The method according to claim 1 wherein the culture is continuous and stable.

14. The method according to claim 13 comprising
a) fermenting the microorganism producing n-butanol;
b) eliminating n-butanol during the fermentation by gas striping; and
c) isolating n-butanol from the condensate by distillation.

15. A microorganism wherein at least one gene involved in butyrate formation is deleted in said microorganism.

16. The microorganism according to claim 15 wherein the deleted gene is at least one of the following genes: ptb encoding phospho-transbutyrylase and buk encoding butyrate kinase.

17. The microorganism according to claim 15 wherein at least one gene involved in acetone formation is attenuated.

18. The microorganism according to claim 17 wherein at least one of the following genes is deleted: ctfAB encoding CoA-transferase and adc encoding aceto-acetate decarboxylase.

19. The microorganism according to claim 15 wherein it is modified to be unable to produce lactate.

20. The microorganism according to claim 19 wherein the ldh gene is deleted.

21. The microorganism according to claim 15 wherein it is modified to be unable to produce acetate.

22. The microorganism according to claim 15 in which at least one gene involved in acetate formation is deleted.

23. The microorganism according to claim 22 wherein the deleted gene is pta encoding phospho-transacetylase or ack encoding acetate kinase.

24. The microorganism according to claim 15 wherein the hydrogen flux is decreased and the reducing power redirected to butanol production.

25. The microorganism according to claim 24 wherein the hydA gene is attenuated.

26. The microorganism according to claim 15 wherein it is C. acetobutylicum, C. beijerinckii, C. saccharoperbutylacetonicum or C. saccharobutylicum.
